(19) United States
(12) Patent Application Publication
(10) Pub. No.: US 2010/0285087 A1
(43) Pub. Date: Nov. 11, 2010

(54) SINGLE PHENOTHIAZINE ENANTIOMERS AS AGENTS FOR THE PREVENTION OF BONE LOSS

(75) Inventors: Joseph McDonough, Helotes, TX (US); Stanton Furst McHardy, Helotes, TX (US)

Correspondence Address: GIFFORD, KRASS, SPRINKLE,ANDERSON & CITKOWSKI, P.C, PO BOX 7021, TROY, MI, 48007-7021, US

(73) Assignee: The University Of Texas, San Antonio, TX (US)

(21) Appl. No.: 12/697,512

(22) Filed: Feb. 1, 2010

Related U.S. Application Data
(63) Continuation-in-part of application No. 10/556,878, filed on May 10, 2006.
(60) Provisional application No. 60/471,155, filed on May 16, 2003.

(51) Int. Cl. A61K 31/542; C07D 279/26; A61K 31/5415; C07D 279/22; C07D 513/04; A61F 2/28; A61K 9/70; A61P 19/04

(52) U.S. Cl. 424/423; 544/41; 514/226.2; 514/225.8; 544/38; 514/224.8; 544/34; 514/224.5; 424/449

(57) ABSTRACT

Enantomerically purified phenothiazines are provided as active ingredients of medicaments to limit activity of bone resorbing cells so as to reduce bone loss. Novel phenothiazine derivatives are provided. A method of synthesizing enantiomerically pure phenothiazine derivatives is provided that avoids post-synthetic enantiomeric resolution.

### CROSS-REFERENCE TO RELATED APPLICATIONS

This application is a continuation-in-part of U.S. application Ser. No. 10/556,878 filed May 5, 2006, which is the U.S. national phase of PCT Application No. PCT/US04/15416 filed May 17, 2004, which claims priority of U.S. Provisional Application No. 60/471,155 filed May 16, 2003, the entire disclosure of which application is specifically incorporated herein by reference without disclaimer. Applicants reserve the right to claim priority to co-pending PCT Application No. PCT/US03/02797, filed Jan. 31, 2003, and to U.S. Provisional Application No. 60/353,633, filed Jan. 31, 2002 and to U.S. Provisional Application No. 60/353,766, filed Jan. 31, 2002; the entire disclosures of which applications are specifically incorporated herein by reference without disclaimer irrespective of reserving the right to claim priority thereto.

### BACKGROUND OF THE INVENTION

1. Field of the Invention

The present invention relates generally to the fields of osteoclasts and bone. The invention provides surprisingly effective methods for inhibiting osteoclasts, reducing bone loss and treating conditions such as periodontitis and osteoporosis by administering a phenothiazine purified enantiomer and associated medicaments, compositions and kits.

2. Description of Related Art

The two major cell types that form and degrade bone are the osteoblast and the osteoclast, respectively. The improper functioning of such cells can produce aberrant bone metabolism, which is linked to the development of several human diseases and disorders, e.g., periodontitis and osteoporosis.

Osteoporosis is defined as compromised bone strength that leads to an increased risk of fracture. This condition results from the mis-regulation of the osteoblast and the osteoclast, thus disturbing the balance of bone formation and degradation.

Osteoporosis is a significant problem in the elderly, in individuals with genetic defects and in those who undergo prolonged space flight. In the weightless environment, bone loss occurs at approximately 2% per month, due to decreased osteoblast activity without alteration in osteoclast activity. Post-menopausal osteoporosis poses a considerable health problem. Significant bone loss in women occurs following estrogen removal, which is due to an increase in osteoclastic activity.

In the United States, osteoporosis is responsible for more than 1.5 million factures annually, including 300,000 hip fractures, 700,000 vertebral fractures, 250,000 wrist fractures and 300,000 fractures at other sites. The estimated national direct expenditure (hospitals and nursing homes) for osteoporotic and associated fractures was $17 billion in 2001, and the cost is rising.

Although there is no cure for osteoporosis, the following medications are approved by the FDA for postmenopausal women to prevent and/or treat osteoporosis: bisphosphonates, such as alendronate (brand name Fosamax®C), risedronate (brand name Actonel®); calcitonin (brand name Miacalcin®); estrogen/hormone replacement therapy, including estrogens (brand names, such as Climara®, Estrace®, Estraderm®, Estratab®, Ogen®, Premaring and others) and estrogens and progestins (brand names, such as Activella®, FemHrt®, Premphase®, Prempro® and others) and selective estrogen receptor modulators (SERMs), such as Raloxifene (brand name Evista®).

Unfortunately, all current treatment modalities suffer from certain drawbacks. For example, the available medications are all expensive and problematical to dose. Importantly, most of the current treatments have serious side effects that result in secondary problems. For example, estrogen treatment is associated with increased risk of cancers in females.

Accordingly, there remains in the art a need for improved methods for preventing or treating bone loss, particularly bone loss associated with osteoporosis. The development of methods to prevent or reduce bone loss with higher efficacy and lower risk of side effects is highly desirable, particularly if the methods could be developed at relatively low cost.

### SUMMARY OF THE INVENTION

The present invention overcomes the foregoing deficiencies and solves certain long-felt needs in the art by providing improved uses and methods for preventing or treating bone loss, such as bone loss associated with periodontitis and osteoporosis. The invention particularly provides uses, medicaments and methods for reducing bone loss and, e.g., treating periodontitis and osteoporosis, by administering a biologically or therapeutically effective amount of only one enantiomer of a chiral phenothiazine or promethazine derivatives. By way of example, the (+) enantiomer of promethazine, for example, has been found to be surprisingly effective in comparison to the racemate and the (−) enantiomer.

Use of the phenothiazine and promethazine enantiomers according to the invention is believed to exert beneficial and anti-osteoporotic effects via osteoclast antagonism or inhibition, The invention thus provides methods of antagonizing or inhibiting osteoclasts by providing an effective inhibitory amount of a purified phenothiazine enantiomer or derivative. Preferably, the (+) enantiomer of promethazine (typically promethazine hydrochloride) or promethazine derivatives is used. The invention further provides phenothiazine and promethazine purified enantiomers modified to increase their hydrophilic characteristic, thereby decreasing their blood-brain barrier penetration.

The effective inhibitory amounts and biologically and therapeutically effective amounts are amounts effective to inhibit osteoclasts and, preferably, amounts effective to reduce bone loss, thereby treating or preventing osteoporosis and other diseases related to bone loss, such as Paget's disease (osteitis deformans) and periodontal disease. Important advantages of the invention include high efficacy and decreased negative indications in comparison to the current methods for treating osteoporosis, as well as the low cost of treatment.

The uses, methods, medicaments, compositions, kits and combinations encompassed by the invention, and various exemplary and certain preferred embodiments thereof, are described in the present specification and the appended claims.

In particular embodiments, the invention provides methods of inhibiting osteoclasts, comprising contacting a population of cells comprising osteoclasts with a biologically effective amount of a composition comprising a substantially purified enantiomer of a phenothiazine chiral phenothiazine derivative.

Although the invention has significant in vivo uses, particularly as the racemic mixtures are currently used in the clinic, the invention also has in vitro uses in the inhibition of osteoclasts in cell populations and tissue samples ex vivo. For example, in maintaining the integrity of bone tissue samples prior to use in bone grafts and such like.

Accordingly, the invention provides methods of inhibiting osteoclasts, comprising contacting a population of cells comprising osteoclasts in vitro with a biologically effective amount of a composition comprising a substantially purified enantiomer of a chiral phenothiazine. The population of cells comprising osteoclasts may be a population of cultured cells or an in vitro bone tissue sample.

Where the population of cells comprising osteoclasts is a substantially intact bone tissue sample, such a substantially intact bone tissue sample may thus be an in vitro bone tissue sample or a bone tissue sample within an animal or patient in vivo. In embodiments where the population of cells comprising osteoclasts is located within an animal, the composition comprising a biologically effective amount of a substantially purified enantiomer of a chiral phenothiazine or a substituted promethazine derivative, is administered to the animal or patient, thereby contacting the cell population or tissue sample in situ.

The invention further includes methods of, and uses in, inhibiting osteoclastic resorption of bone, bone tissue and bone tissue samples. The methods generally comprise contacting a bone tissue sample with a composition comprising a substantially purified enantiomer of a chiral phenothiazine, preferably the (+) enantiomer of promethazine or a substituted promethazine derivative, in an amount effective to inhibit osteoclasts in the bone tissue sample, thereby inhibiting osteoclastic resorption of bone tissue.

Again, the bone tissue sample may be an in vitro bone tissue sample or a bone tissue sample within an animal or patient in vivo. Where the bone tissue sample is maintained ex vivo or in vitro, the methods comprise contacting an in vitro bone tissue sample with the chiral phenothiazine composition in an amount effective to inhibit osteoclasts in the bone tissue sample, thereby inhibiting osteoclastic resorption of bone tissue in vitro.

Where the bone tissue sample is located within an animal, the methods comprise providing to an animal or patient exhibiting or at risk for osteoclastic resorption of bone, a composition comprising a substantially purified enantiomer of a chiral phenothiazine, preferably the (+) enantiomer of promethazine or a substituted promethazine derivative, in an amount effective to inhibit osteoclasts in the animal or patient, thereby inhibiting osteoclastic resorption of bone in the animal or patient.

Important aspects of the invention are methods of, and uses in, treating or preventing bone loss. Methods of treating or preventing bone loss generally comprise providing to an animal or patient exhibiting or at risk for bone loss a biologically or therapeutically effective amount of a composition comprising a substantially purified enantiomer of a chiral phenothiazine, preferably the (+) enantiomer of promethazine or a substituted promethazine derivative.

Similar embodiments of the invention concern use of a composition comprising a substantially purified enantiomer of a chiral phenothiazine, preferably the (+) enantiomer of promethazine or a substituted promethazine derivative, in the manufacture of a medicament for use in inhibiting osteoclasts for treating or preventing a disease or condition associated with bone loss.

Other methods of the invention are methods of reducing bone loss, comprising providing to an animal or patient exhibiting bone loss an amount of a composition comprising a substantially purified enantiomer of a chiral phenothiazine, preferably the (+) enantiomer of promethazine or a substituted promethazine derivative, effective to treat bone loss in the animal or patient.

Related uses of the invention concern the manufacture of a medicament for use in inhibiting osteoclastic resorption of bone, the manufacture of a medicament for treating or preventing bone loss, and the manufacture of a medicament for treating bone loss. A particular use of the invention is the use of a composition comprising a substantially purified enantiomer of a chiral phenothiazine, preferably the (+) enantiomer of promethazine or a substituted promethazine derivative, in the manufacture of a medicament for use in treating or preventing a disease or condition associated with bone loss.

In the methods and uses of the invention, the animal or patient may have or be at risk for developing periodontitis, including chronic destructive periodontal disease. Accordingly, the invention provides methods of treating or preventing periodontitis, comprising providing to an animal or patient having or at risk for developing periodontitis a therapeutically effective amount of a composition comprising a substantially purified enantiomer of a chiral phenothiazine, preferably the (+) enantiomer of promethazine or a substituted promethazine derivative. In the treatment methods, the methods comprise providing to an animal or patient with periodontitis a therapeutically effective amount of a composition comprising the chiral phenothiazine, preferably a substantially purified (+) enantiomer of promethazine. In such methods, the biologically and therapeutically effective amounts are typically amounts effective to treat periodontitis upon administration of the composition to the animal or patient.

Related uses of the invention concern the manufacture of a medicament for use in treating or preventing periodontitis or chronic destructive periodontal disease and the manufacture of a medicament for treating periodontitis or chronic destructive periodontal disease.

The animal or patient to be treated by the methods and uses of the invention may have or be at risk for developing osteoporosis. The invention thus provides methods of treating or preventing osteoporosis, comprising providing to an animal or patient having or at risk for developing osteoporosis a therapeutically effective amount of a composition comprising a substantially purified enantiomer of a chiral phenothiazine, preferably the (+) enantiomer of promethazine or a substituted promethazine derivative. In the treatment methods, the methods comprise providing to an animal or patient with osteoporosis a therapeutically effective amount of a composition comprising the chiral phenothiazine, preferably a substantially purified (+) enantiomer of promethazine.

In such methods, the biologically and therapeutically effective amounts are typically amounts effective to treat osteoporosis upon administration of the composition to the animal or patient. Such “an effective amount” is therefore termed “an anti-osteoporotic amount”.

Although the treatment of osteoporosis in all settings is encompassed by the invention, the treatment of osteoporosis is particularly important in post-menopausal females, which therefore form a preferred treatment group within the methods and uses of the invention.

Related uses of the invention concern the manufacture of a medicament for use in treating or preventing osteoporosis, the manufacture of a medicament for treating osteoporosis and the manufacture of a medicament for treating osteoporosis in post-menopausal females.

The invention further provides kits and therapeutic kits, which generally comprise, in at least a first container:
(a) a biologically or therapeutically effective amount of a substantially purified enantiomer of a chiral phenothiazine, preferably the (+) enantiomer of promethazine or a substituted promethazine derivative; and
(b) a biologically or therapeutically effective amount of a second, distinct anti-osteoclastic or anti-osteoporotic agent.

The biologically or therapeutically effective amounts of the chiral phenothiazines of the kits are typically amounts effective to inhibit osteoclasts, inhibit bone resorption, treat or prevent periodontitis, and/or treat or prevent osteoporosis and such like. In the kits, the chiral phenothiazine and the second, distinct anti-osteoclastic or anti-osteoporotic agent may be comprised within a single container, such as an admixture, or may be comprised separately, within distinct containers.

The kits of the invention may further comprise instructions for using the kits to inhibit osteoclasts, inhibit bone resorption, treat or prevent periodontitis, treat or prevent osteoporosis and/or manufacture a medicament for use in any such preventative or treatment modalities. The instructions may be written, or in electronic or computerized form.

In each of the uses, methods, medicaments, compositions, kits and combinations of the invention, the use of an enantiomerically pure enantiomer of a chiral phenothiazine is preferred. A range of chiral phenothiazines may be used in the invention, such as wherein the chiral phenothiazine has the structure:

where

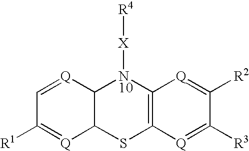

R¹, R², and R³ are each independently H, F, Cl, Br, I, C₁-C₆ alkyl, C₁-C₆ fluoro-alkyl, C₁-C₆ perfluoro-alkyl, (C₀-C₆ linear alkyl or C₀-C₆ branched alkyl)-O—R⁶, (C₀-C₆ linear alkyl or C₀-C₆ branched alkyl)-R⁶—OH, (C₀-C₆ linear alkyl or C₀-C₆ branched alkyl)-C(O)OR⁶, —C(O)—R⁶ alkyl, —(C₀-C₆ linear alkyl or C₀-C₆ branched alkyl)-P(O)—(O—R⁶)2, (C₀-C₆ linear alkyl or C₀-C₆ branched alkyl)-S(O)₂—R⁶, (C₀-C₆ linear alkyl or C₀-C₆ branched alkyl)—N(R⁶)₂, (C₀-C₆ linear alkyl or C₀-C₆ branched alkyl)-N⁺(R⁶)₃, (C₀-C₆ alkyl)-P⁺R₄, (C₀-C₆ alkyl)-S⁺(R⁶)₂,

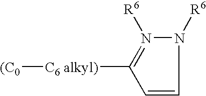

C₆-aryl, a substituted C₆-aryl where the substituent is at least one of hydroxyl radicals, halogens, alkyl radicals having a total of from 1 to 6 carbon atoms, cyclic alkylene groups and heterocyclic alkylene groups having a heterocyclic element of nitrogen and sulfur where C₀ alkyl denotes a nullity;

X is a C₁-C₅ linear alkyl, C₁-C₅ branched alkyl, a C₁-C₅ linear alkenyl, or C₁-0₅ branched alkenyl with the proviso that at least one chiral carbon atom is present in X and the compound is a purified enantiomer of the at least one chiral carbon atom present in X when R² is other than (C₀-C₆ linear or branched alkyl)-C(O)OR⁶ or (C₀-C₆ linear or branched alkyl)-S(O)₂—R⁶;

R⁴ is a tertiary amine or thiol radical structure of N—(R⁵)₂ or S—R⁵ wherein R⁵ in each occurrence is independently hydrogen, C₁-C₄ alkyl radicals and C₁-C₄ alkenyl radical having cyclic alkylene groups or heterocyclic alkylene groups having a heterocyclic element of nitrogen or sulfur;

R⁶ is independently in each occurrence H, C₁-C₄ alkyl and N—(R⁵)₂; and

Q is independently in each occurrence an sp²-hybridized C—R⁶ or a nitrogen atom.

In particular embodiments, R⁵ is selected from the group consisting of alkyl radicals and alkenyl radical having from about 1 to about 3 carbon atoms.

Particular chiral purified phenothiazines about the at least one chiral carbon atom of X for use in the invention are promethazine, ethopropazine, propiomazine and trimeprazine. Where the chiral phenothiazine is an ethopropazine, the invention concerns the use of a substantially purified (−) enantiomer of ethopropazine.

In particularly preferred embodiments, the chiral phenothiazine of the uses, methods, medicaments, compositions, kits and combinations of the invention is a substantially purified (+) enantiomer of promethazine.

A process for synthesizing a compound having an enantiomerically purified chiral carbon atom extending from a ring nitrogen at position 10 of a phenothiazine, azo-azeo-phenothiazine. The structural includes reacting a molecule containing the chiral carbon atom as a chloride under alkylation conditions with a phenothiazine having the structure:

where

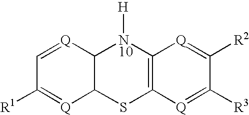

R¹, R², and R³ are each independently H, F, Cl, Br, I, C₁-C₆ alkyl, C₁-C₆ fluoro-alkyl, C₁-C₆ perfluoro-alkyl, (C₀-C₆ linear alkyl or C₀-C₆ branched alkyl)-O—R⁶, (C₀-C₆ linear alkyl or C₀-C₆ branched alkyl)-R⁶—OH, (C₀-C₆ linear alkyl or C₀-C₆ branched alkyl)-C(O)OR⁶, —C(O)—R⁶ alkyl, —(C₆-C₆ linear alkyl or C₆-C₆ branched alkyl)-P(O)-(O-R⁶)2, (C₀-C₆ linear alkyl or C₀-C₆ branched alkyl)-S(O)₂—R⁶, (C₀-C₆ linear alkyl or C₀-C₆ branched alkyl)-N(R⁶)₂, (C₀-C₆ linear alkyl or C₀-C₆ branched alkyl)-N⁺(R⁶)₃, (C₀-C₆ alkyl)-P⁺R₄, (C₀-C₆ linear alkyl or C₆-C₆ branched alkyl)-S⁺(R⁶)₂,

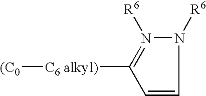

C₆-aryl, a substituted C₆-aryl where the substituent is at least one of hydroxyl radicals, halogens, alkyl radicals having a total of from 1 to 6 carbon atoms, cyclic alkylene groups and heterocyclic alkylene groups having a heterocyclic element of nitrogen and sulfur where C₀ alkyl denotes a nullity;

X is a C₁-0₅ or branched alkyl or a C₁-C₅ or branched alkenyl with the proviso that at least one chiral carbon atom is present in X;

a purified enantiomer of the at least one chiral carbon atom present in X when R² is other than (C₀-C₆ linear alkyl or C₀-C₆ branched alkyl)-S(O)₂—R⁶;

R⁴ is a tertiary amine or thiol radical structure of N—(R⁵)₂ or S—R⁵ wherein R⁵ in each occurrence is independently hydrogen, C₁-C₄ alkyl radicals and C₁-C₄ alkenyl radical having cyclic alkylene groups, or heterocyclic alkylene groups having a heterocyclic element of nitrogen or sulfur;

R⁶ is independently in each occurrence H, C₁-C₄ alkyl and N—(R⁵)₂; and

Q is independently in each occurrence an sp²-hybridized C—R⁶ or a nitrogen atom to produce the compound having the enantiomerically pure chiral carbon atom extending from the ring nitrogen atom at position 10.

### BRIEF DESCRIPTION OF THE DRAWINGS

The following drawings form part of the present specification and are included to further demonstrate certain aspects of the present invention. The invention may be better understood by reference to the drawings in combination with the detailed description of specific embodiments presented herein.

FIG. 1. Bone is a complex, highly regulated tissue composed of cells and calcified matrix.

FIG. 2. A racemic mixture of promethazine inhibit bone resorption in vitro (taken from Hall and Schaueblin, 1994).

FIG. 3. Osteoclast physiology and biology.

FIG. 4. Effect of phenothiazine analogues on osteoclastic bone resorption. The compounds were added at the start of the 24-h bone slice assay. The results shown are the mean ±SEM of three separate studies for each compound tested (15 bone slices per point). The control area resorbed per bone slice value (mean±SEM) for a total of 15 studies was 7570±910 m² (taken from Schaeublin et al., 1996).

FIG. 5A, FIG. 5B, FIG. 5C and FIG. 5D. Effects of the racemic mixture and the (+) and (−) enantiomers of promethazine on osteoclast resorption at 10⁻⁵ M, 10⁻⁶ M and 10⁻⁷ M in controlled studies using an osteoclast resorption assay.

FIG. 6. Quantification of the effects of the racemic mixture (R) and the (+) and (−) enantiomers of promethazine in terms of absorption area per slice at 10⁻⁵ M, 10⁻⁶ M and 10⁻⁷ M. n=12,* p<0.05,**p<0.01.

FIG. 7 is a synthetic saline for inventive chiral phenothiazine derivatives.

FIG. 8 is an alternate synthetic scheme for inventive chiral phenothiazine derivatives having heterocyclic moieties and secondary and tertiary hydroxymethyl moieties extending from the conjugate ring system core.

FIG. 9 is a synthetic scheme for inventive chiral phenothiazine derivatives having a keto, carboxyl, or ester moiety extending from the conjugate ring system core.

FIG. 10 is an alternate synthetic scheme having a sulfonamide moiety extending from the conjugate ring system core.

FIG. 11 is an alternate synthetic scheme having an azo-phenothiazine conjugated ring system core.

FIG. 12 is still another alternate synthetic scheme having an azo-phenothiazine conjugated ring system core.

FIG. 13 is a control bar graph for alendronate (bis-phosphonate) showing calcium release as a result of osteoclast-mediated resorptive activity as a function of concentration with statistical error bars provided.

FIG. 14A is a bar graph for (S)-N,N-dimethyl-1-(10H-phenothiazin-10-yl)propan-2-amine showing calcium release as a result of osteoclast-mediated resorptive activity as a function of concentration with statistical error bars provided.

FIG. 14B is a bar graph for (R)-N,N-dimethyl-1-(10H-phenothiazin-10-yl)propan-2-amine showing calcium release as a result of osteoclast-mediated resorptive activity as a function of concentration with statistical error bars provided.

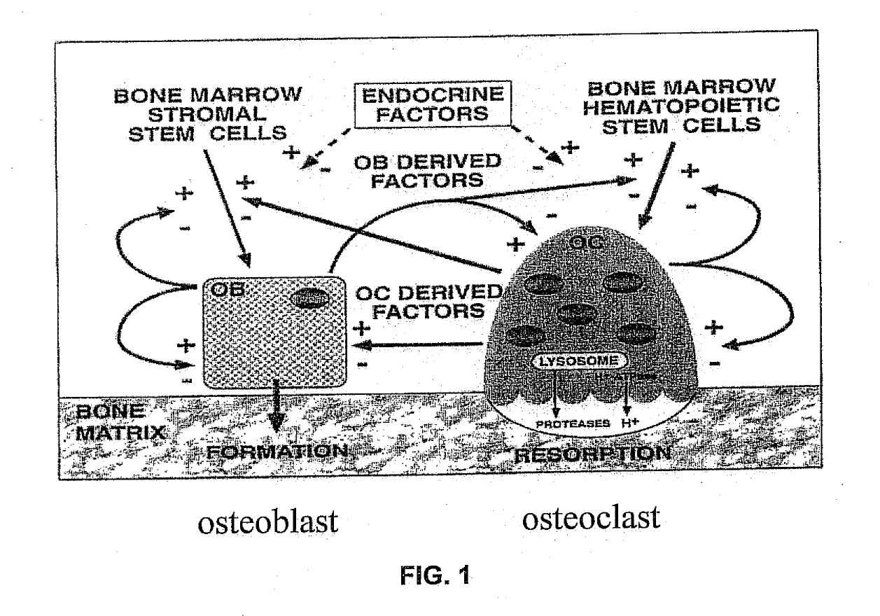

### DESCRIPTION OF ILLUSTRATIVE EMBODIMENTS

In adults, bone tissue undergoes a continual process of renewal, which is orchestrated by osteoblasts and osteoclasts, specialized cell types that originate from progenitors residing in the bone marrow. The numbers and activity of osteoblasts and osteoclasts is important for bone homeostasis, as changes in their number and activity result in imbalances between bone formation and bone resorption, which can lead to systemic and/or localized bone diseases, including periodontitis and osteoporosis.

Bone mass can be increased by intermittent parathyroid hormone (PTH) administration. It has been reported that daily PTH injections in mice with either normal bone mass or osteopenia due to defective osteoblastogenesis increased bone formation without affecting the generation of new osteoblasts (Jilka et al., 1999). These authors reported that PTH increased the life-span of mature osteoblasts by preventing their apoptosis; and the anti-apoptotic effect was said to be sufficient to account for the increase in bone mass, as confirmed in vitro using rodent and human osteoblasts and osteocytes (Jilka et al., 1999). Jilka et al. (1999) thus suggested the use of anti-apoptotic strategies to treat osteoporosis and other pathological conditions in which bone mass diminution compromises functional integrity.

Periodontitis can result when inflammation or infection of the gums (gingivitis) is untreated and/or treatment is delayed. Infection and inflammation typically spreads from the gums (gingiva) to the ligaments and bone that support the teeth. Loss of support causes the teeth to become loose and eventually fall out. Periodontitis is the primary cause of tooth loss in adults,

During periodontitis, plaque and tartar accumulate at the base of the teeth, Inflammation causes a pocket to develop between the gums and the teeth, which fills with plaque and tartar. Soft tissue swelling traps the plaque in the pocket. Continued inflammation causes destruction of the tissues and bone surrounding the tooth. Bacteria in plaque cause infection, which can develop into a tooth abscess, further increasing the rate of bone destruction,

In addition to the use of PTH to augment bone mass in osteoporosis and associated conditions, it has recently been reported that PTH protects against periodontitis-associated bone loss (Barros et al., 2003). In these studies, Barros et al., (2003) demonstrated that intermittent administration of PTH blocked the alveolar bone loss observed in rats in a ligature model of periodontitis. These studies therefore show that restoring the balance between osteoblasts and osteoclasts, in this case by inhibiting apoptosis of osteoblasts, is effective in treating periodontitis.

The bisphosphonate compound, incadronate (YM175, disodium cycloheptylamino-methylenediphosphonate monohydrate), has been suggested to prevent the bone resorption associated with periodontitis by inhibiting osteoclast activity. Tani-Ishii et al., (2003) recently reported that incadronate was effective in preventing periodontal destruction in rats with Porphyromonas gingivalis-induced periodontitis. In controlled studies, it was shown that incadronate increased the bone mineral density and prevented periodontal ligament destruction in rats with P. gingivalis infections (Tani-Ishii et al., 2003). These studies therefore complement those described above, showing that compounds that inhibit osteoclast activity are able to treat periodontitis. Unfortunately, as with osteoporosis, the agents currently available for use in such treatment all suffer from certain drawbacks, including serious side effects.

Whilst periodontitis takes a toll in healthcare in the developed world, osteoporosis is a very significant problem. In the United States, the estimated national direct expenditures for osteoporotic and associated fractures were $17 billion in 2001. One of the agents approved for use in treating osteoporosis is promethazine (PMZ). In administering promethazine to human subjects, Tyan (1993) observed a 3% annual gain in spinal bone mass in post-menopausal women with a dosage of 50 mg/day promethazine associated with estrogens. Rico et al. (1999) also reported on the effects of promethazine on bone loss in rats (Table 1, taken from Table 1 in Rico et al., 1999).

TABLE 1
The Effects of Promethazine on Bone Loss in Rat

| | Controls | OVX | OVX + PROM | Sham-OVX |
|---|---|---|---|---|
| N | 15 | 15 | 15 | 15 |
| Initial | 242 ± 28 | 252 ± 26 | 253 ± 17 | 257 ± 12 |
| weight g | | | | |
| Final | 305 ± 22 | 327 ± 31^{a} | 318 ± 26 | 293 ± 18 |
| weight g | | | | |
| Femur mm | 33.6 ± 0.4 | 33.8 ± 1.1 | 34.5 ± 0.7 | 34.0 ± 0.7 |
| Femur mg | 720 ± 52 | 679 ± 68 | 716 ± 38 | 704 ± 41 |
| F-BMC mg | 390 ± 31 | 292 ± 36^{b} | 336 ± 30 | 346 ± 21 |
| Z score | 0.1 ± 0.9 | −3.1 ± 1.1^{b} | −1.7 ± 0.9 | −1.4 ± 0.6 |
| F-BMC | | | | |
| F-BMD | 135 ± 11 | 113 ± 13^{c} | 125 ± 14 | 125 ± 9 |
| mg/cm² | | | | |
| F-BMC/BW | 1.28 ± 0.03 | 0.90 ± 0.09 | 1.06 ± 0.06 | 1.18 ± 0.07 |
| mg/g | | | | |
| Vertebra mm | 6.2 ± 0.2 | 6.2 ± 0.6 | 6.3 ± 0.3 | 6.1 ± 0.2 |
| Vertebra mg | 249 ± 14 | 217 ± 31^{d} | 253 ± 24 | 241 ± 23 |
| V-BMC mg | 116 ± 13 | 86 ± 18^{d} | 100 ± 10 | 111 ± 12 |
| V-BMD | 111 ± 8 | 101 ± 8^{d} | 108 ± 6 | 112 ± 7 |
| mg/cm² | | | | |
| Cn-BV- | 19.9 ± 6.6 | 8.6 ± 4.1^{c} | 12.5 ± 5.6 | 24.2 ± 5.9 |
| TV % | | | | |
| Tb-N mm⁻¹ | 2.9 ± 0.6 | 1.6 ± 0.6^{c} | 2.1 ± 0.7 | 3.0 ± 0.2 |
| Tb-Th μm | 68.2 ± 19.6 | 52.3 ± 9.0 | 57.5 ± 7.3 | 70.1 ± 16.6 |
| Tb-Sp μm | 285 ± 70 | 857 ± 281^{c} | 477 ± 215 | 253 ± 38 |

Femur bone mineral content and density (F-BMC mg and F-BMD mg/cm²);
5^{th} lumbar vertebral bone mineral content and density (V-BMC mg and V-BMD mg/cm²);
femur cancellous bone volume (Cn-BV/TV %);
trabecular number (Tb-N mm⁻¹);
trabecular thickness (Tb-Th μm); and
trabecular separation (Tb-Sp μm);
ovariectomized rats (OVX);
promethazine-treated ovariectomized rats (OVX + PROM); and
untreated Sham-ovariectomized rats (Sham-OVX),
^{a}P = 0.001 vs. Sham-OVX;
^{b}P < 0.0001 vs. others;
^{c}P < 0.0005 vs. others;
^{d}P < 0.0005 vs. OVX + PROM.
According to ANOVA with post hoc Bonferroni-Dunn test.

Taken from Table 1 in Rico et al., 1999, specifically incorporated herein by reference.

Promethazine is also used as an anti-emetic, anti-histamine and anti-psychotic agent. The predominant mode of action is antagonism of histamine receptors. There are different histamine receptors, including histamine receptor 1 (H₁) and histamine receptor 2 (H₂). Promethazine is a member of the phenothiazines. Primarily anti-histamines, these compounds are promiscuous drugs and have been shown to be antagonists of the muscarinic cholinergic and dopamine receptors, albeit with lower affinity. Sedation is significant at concentrations achieved from therapeutic dosages. The sedative effects of promethazine require direct CNS interaction, FDA approval for promethazine was issued in 1954.

U.S. Pat. No. 4,256,743, specifically incorporated herein by reference, concerns methods for inhibiting bone resorption by orally administering H₁-blocking phenothiazines, including promethazine hydrochloride. In particular, the patent describes methods for inhibiting bone resorption in mammals suffering from osteoporosis or chronic destructive periodontal disease by administering specified amounts of an H₁-blocking anti-histamine compound (H₁ antagonist) in the form of an H₁-blocking phenothiazine, H₁-blocking ethylenediamine or H₁-blocking indene. The amounts specified for H₁-blocking phenothiazines are 25-50 mg/day.

In developing the present invention, the implications resulting from the fact that promethazine is chiral are considered. “Chiral” or optically active compounds exist in two forms: the (+) form and the (−) form. The (+) and (−) descriptors relate to how the enantiomers rotate the plane of plane-polarized light. The particular forms of chiral compounds are termed stereoisomers or enantiomers. A combination of the two forms is termed the racemate or racemic mixture.

Prior to the present invention, the clinical uses of promethazine, including the use of promethazine as an osteoporosis inhibitor, as in U.S. Pat. No. 4,256,743, utilized the compound as a racemic mixture. Indeed, the (+) form and the (−) form have been shown to be present at a ratio of 50:50 in clinical samples. The histamine receptor is chiral sensitive. Enantiomers of promethazine have been described in the literature in reference to combination pharmaceuticals (U.S. Pat. No. 6,103,735; U.S. patent application 2002/0151541; and U.S. patent application 2002/0198231). Such combined pharmaceuticals have been proposed for use in treating asthma and allergic conditions, respiratory tract diseases and Parkinson's disease. However, prior to the present invention, there was no information concerning the chiral sensitivity of promethazine's effects on osteoclasts.

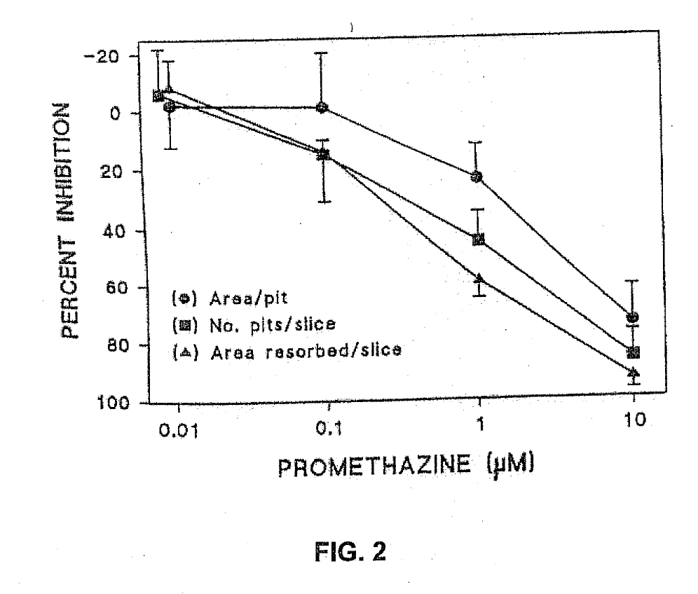

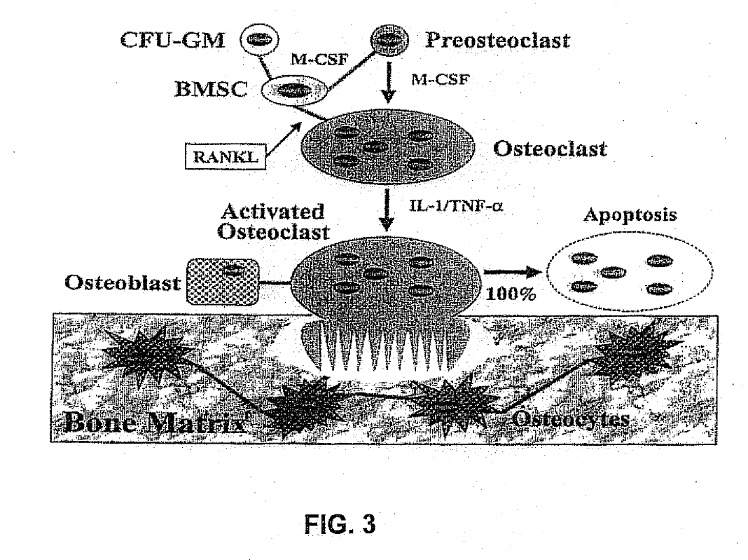

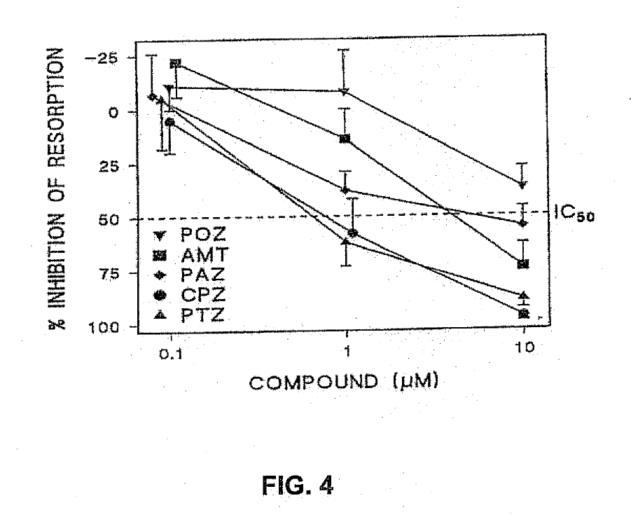

In vitro, increasing concentrations of promethazine had been shown to inhibit bone resorption (FIG. 2, from Hall and Schaueblin, 1994). Osteoporosis results from the dysregulation of osteoblasts, cells that form bone, and osteoclasts, cells that degrade bone (FIG. 3). Dobigny and Safar (1997) reported on the anti-histamine actions of certain H₁ receptor antagonists and H₂ receptor antagonists (Table 2, taken from Table 1 in Dobigny and Safar, 1997). As to the non-anti-histamine actions, Schaeublin et al. (1996) reported the effect of phenothiazine analogues on osteoclastic bone resorption in a 24-h bone slice assay (FIG. 4, from Schaeublin et al., 1996).

TABLE 2
Anti-Histamine Actions of H₁ and H₂ Receptor Antagonists

| | | Total | Active | Inactive | |
|---|---|---|---|---|---|
| | RS | Osteoclasts | Osteoclasts | Osteoclasts | OBIT |
| Sham (n = 8) | 32.4 ± 2.7 | 19.1 ± 1.9 | 14.1 ± 1.3 | 5.0 ± 0.7 | 23.3 ± 1.1 |
| Mepyramine (n = 8) | 17.6 ± 1.3*** | 15.3 ± 1.5 | 9.6 ± 0.7* | 5.7 ± 0.8 | 18.4 ± 0.8** |
| Cimetidine (n = 8) | 14.4 ± 2.0** | 9.4 ± 1.4**,**** | 6.2 ± 0.8***,***** | 3.1 ± 0.7 | 24.7 ± .9*,**** |
| Kruskal-Wallis test | H = 15.39 | H = 11.19 | H = 16.57 | H = 4.41 | H = 7.70 |
| | P = 0.005 | P = 0.0032 | P = 0.0003 | NS | P = 0.0212 |

The table shows modifications of resorption parameters induced by mepyramine (H₁ receptor antagonist) and cimetidine (H₂ receptor antagonist) at the peak of resorption. The peak of bone resorption is 4 days in the synchronized model of # resorption used; active osteoclasts are on-bone cells, and inactive osteoclasts are off-bone cells. OBI is the mean interface of the active cells with the bone surface.
Group comparisons were performed by using the Mann-Whitney U Test.
*Different from controls, P < 0.05.
**Different from controls, P < 0.01.
***Different from controls, P < 0.001.
****Different from the mepyramine group, P < 0.05.
*****Different from the mepyramine group, P < 0.01.

Taken from Table 1 in Dobigny and Safar, 1997, specifically incorporated herein by reference.

From these observations, for the H₁ antagonists, the non-histamine pathway appears to be the primary pathway. Thus, promethazine primarily inhibits osteoclastic resorption of bone via a histamine independent pathway; although a histamine dependent pathway may also be involved. As the mechanism of action of promethazine in the present invention is primarily independent of histamine inhibition, it is thus different to that underlying U.S. Pat. No 4,256,743.

The chiral nature of the promethazine molecule suggested that only one of the enantiomers may be responsible for the biological activity observed for the racemic mixture. This proved to be the case, and the present application shows that activity resides in the (+) enantiomer and, surprisingly, that the (+) enantiomer has a three fold higher efficacy for osteoclast inhibition than the racemate and the (−) enantiomer in controlled studies. As used herein, the terms (+) and (−) enantiomer refer to optical rotation as measured in water.

A compound according to the present invention is provided that is a chiral phenothiazine that has the structure:

where

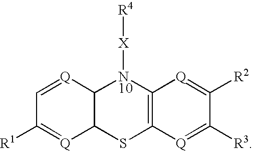

R¹, R², and R³ are each independently H, F, Cl, Br, I, C₁-C₆ alkyl, C₁-C₆ fluoro-alkyl, C₁-C₆ perfluoro-alkyl, (C₀-C₆ linear alkyl or C₀-C₆ branched alkyl)—O—R⁶, (C₀-C₆ linear alkyl or C₀-C₆ branched alkyl)-R⁶—OH, (C₀-C₆ linear alkyl or C₀-C₆ branched alkyl)-C(O)OR⁶, —C(O)—R⁶ alkyl, (C₀-C₆ linear alkyl or C₀-C₆ branched alkyl)-P(O)—(O—R⁶)₂, (C₀-C₆ linear alkyl or C₀-C₆ branched alkyl)-S(O)₂—R⁶, (C₀-C₆ linear alkyl or C₆-C₆ branched alkyl)-N(R⁶)₂, (C₀-C₆ linear alkyl or C₀-C₆ branched alkyl)-N⁺(R⁶)₃, (C₀-C₆ linear alkyl or C₀-C₆ branched alkyl)-P⁺R₄, (C₀-C₆ linear alkyl or C₀-C₆ branched alkyl)-S⁺(R⁶)₂,

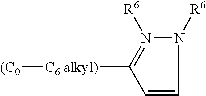

C₆-aryl, a substituted C₆-aryl where the substituent is at least one of hydroxyl radicals, halogens, alkyl radicals having a total of from 1 to 6 carbon atoms, cyclic alkylene groups and heterocyclic alkylene groups having a heterocyclic element of nitrogen and sulfur where C₀ alkyl denotes a nullity;

X is a C₁-C₅ or branched alkyl or a C₁-C₅ or branched alkenyl with the proviso that at least one chiral carbon atom is present in X and the compound is a purified enantiomer of the at least one chiral carbon atom present in X when R² is other than (C₀-C₆ linear alkyl or C₆-C₆ branched alkyl)-C(O)OR⁶ or (C₀-C₆ linear alkyl or C₀-C₆ branched alkyl)-S(O)₂—R⁶;

R⁴ is a tertiary amine or thiol radical structure of N—(R⁵)₂ or S—R⁵ wherein R⁵ in each occurrence is independently hydrogen, C₁-C₄ alkyl radicals and C₁-C₄ alkenyl radical having cyclic alkylene groups or heterocyclic alkylene groups having a heterocyclic element of nitrogen or sulfur;

R⁶ is independently in each occurrence H, C₁-C₄ alkyl and N—(R⁵)₂; and

Q is independently in each occurrence an sp²-hybridized C—R⁶ or a nitrogen atom.

In testing the (+) enantiomer for cytotoxicity, it was determined that it did not have any significant increase in cytotoxicity compared to the racemic mixture (the same was also true for the (−) enantiomer). The increased activity, without any increase in cytotoxicity, therefore makes the (+) enantiomer of promethazine ideal for treatment in place of the racemic mixture. The present invention therefore provides improved methods of inhibiting osteoclasts, reducing bone loss and treating or preventing periodontitis, osteoporosis and associated disorders by administering the (+) enantiomer of promethazine and, in light of the studies herein and the present disclosure, purified enantiomers of other chiral phenothiazines.

Although (+) and (−) are descriptors of how the enantiomers rotate plane polarized light, the terms R and S describe the absolute configuration. Enantiomers are also described using the terms D and L. The R isomer is the +in the case of promethazine. In other chiral phenothiazines in which the structures more closely resemble promethazine, weak R enantiomers are likely to be active, since this part of the molecule seems to influencing binding. As a histamine dependent pathway may also be involved in the inhibition of osteoclastic resorption of bone by phenothiazines, and the inventors determined that the major anti-histamine activity associated with ethopropazine resides in the (−) enantiomer, the (−) enantiomer of ethopropazine is also contemplated for use in the present invention. In any event, in light of the studies herein, particularly the successful working embodiments and the discriminatory assays described in detail, the enantiomer of any phenothiazine with activity in the inhibition of osteoclasts may now be determined and such an enantiomer used in the methods, medicaments, uses, compositions, combinations and kits of the present invention.

Within the substantially purified enantiomer compositions for use in the present invention, those consisting essentially of the active enantiomer will be particularly preferred, up to and including compositions comprising essentially or substantially the enantiomerically pure active enantiomer. In all embodiments, the (+) enantiomer of promethazine is still currently most preferred.

It will be understood that the terms “composition comprising a substantially purified enantiomer”, “composition consisting essentially of an active enantiomer”, “composition comprising essentially or substantially an enantiomerically pure active enantiomer”, “composition comprising an enantiomerically pure active enantiomer of a phenothiazine”, “composition comprising an enantiomerically pure (+) enantiomer of promethazine”, and like terms, as used herein, refer to the composition and purity of the active enantiomer as opposed to the other enantiomer, such as the purity of the (+) enantiomer of promethazine as opposed to the (−) enantiomer of promethazine. Such terms do not exclude the presence of other components in the composition “comprising” the active enantiomer, such as the (+) enantiomer of promethazine, such as pharmaceutical diluents or components or other biological or therapeutic agents, particularly second agents for treating bone loss or osteoporosis.

Substantially enantiomerically pure and enantiomerically pure compounds can be prepared by those of ordinary skill in the art without undue experimentation in light of the present disclosure. U.S. Pat. No. 38334 (re-issue of U.S. Pat. No. 6,020,506) is specifically incorporated herein by reference for the purpose of describing and enabling the preparation of compositions comprising enantiomerically pure compounds per se.

Although phenothiazines and promethazine are approved for human administration, and available commercially, the present inventors generally do not recommend commercially available forms as starting materials. However, if the source of an active agent for use in the invention is a commercial tablet, then the mixture obtained from the tablet should preferably be treated to provide the active ingredient relatively free, preferably substantially free of the non-active components.

Methods of purification will be well known to those of ordinary skill in light of the present disclosure, and may include, e.g., dissolution of the mixture in a solvent and recrystallization. Column chromatography may be used to resolve a phenothiazine racemate into its two enantiomers (Nilsson et al., 1994; specifically incorporated herein by reference). Racemates may also be separated using chromatographic separation, such as gas chromatography (GC) or high performance liquid chromatography (HPLC), such as used in the resolution of promethazine, ethopropazine, trimeprazine and trimpramine enantiomers (Ponder et al., 1995; specifically incorporated herein by reference). Capillary electrophoresis (CE) may also be employed (Wang et al, 2001; specifically incorporated herein by reference). Certain currently preferred methods for resolving the (+) enantiomer and (−) enantiomers of phenothiazines, including promethazine and ethopropazine, are described in the present examples.

Although the mechanism of action of the phenothiazine and promethazine enantiomers in the present invention is primarily independent of histamine inhibition, and thus different to that underlying U.S. Pat. No. 4,256,743, this patent is nonetheless instructive concerning the ability of those of ordinary skill in the art to practice the in vivo therapies of the present invention without undue experimentation, given the teachings of the present disclosure.

In particular, as U.S. Pat. No. 4,256,743 concerns inhibiting bone resorption and treating osteoporosis and related bone and periodontal diseases using racemic mixtures of phenothiazines such as promethazine, and as this invention provides high efficacy enantiomers for use at lower doses, one can now inhibit bone resorption and treat bone diseases, osteoporosis and periodontal diseases using the enantiomers.

In terms of largely prophylactic uses, such as in the prevention of symptoms, or more significant symptoms, in animals and patients with indicators of disease, prophylactic or preventive doses will be employed. In terms of treatment, therapeutic doses will be used. Although achieving symptom-free states can be achieved using the invention, such end points are not a requirement of any treatment according to the present invention.

Certain of the benefits of the invention include the high efficacy, which lowers the doses required, thus reducing the cost of treatment and avoiding potential side-effects. For example, although the sedative effects of promethazine can be a drawback in certain prior art methods of use, such issues are overcome by the high efficacy provided by the invention. Moreover, the inventors further provide modified enantiomers with increased hydrophilicity, which decrease penetration across the blood-brain barrier and ameliorate negative indicators, such as sedative effects, which are mediated via the CNS.

Although the surprising effectiveness of phenothiazine enantiomers, particularly the (+) enantiomer of promethazine, in preventing bone loss could not have been predicted prior to this invention, the FDA approval for the clinical use of racemic mixtures of these drugs also provides particular advantages, shortening the path to approval for treating osteoporosis using the enantiomers, thus further lowing the cost of treatment.

Any effective means to administer or deliver the phenothiazine enantiomers, preferably the (+) enantiomer of promethazine, may be used in the methods of the present invention, i.e., in the treatment of bone loss and related diseases, such as osteoporosis, Paget's Disease and periodontitis. Preferably, it is currently believed that delivery modes to be avoided are those that favor delivery to the CNS, such as those described in U.S. Pat. No. 5,601,835; U.S. Pat. No. 5,114,719 and U.S. Pat. No. 4,883,666.

One currently preferred delivery method concerns the impregnation of the enantiomeric agents into a resorbable or non resorbable implantable matrix or transdermal delivery device that is conducive to nominal zero order release. Such matrices or delivery devices result in a substantially constant level of drug at a lower plasma concentration, thus achieving the therapeutic benefits whilst even further circumventing any possible adverse side effects. Controlled release oral formulations are also contemplated, but will typically require a higher dose at administration.

The following examples are included to demonstrate certain preferred embodiments of the invention. It will be appreciated by those of skill in the art that the compositions and techniques disclosed in the examples that follow represent compositions and techniques discovered by the inventors to function well in the practice of the invention, and thus can be considered to constitute certain preferred modes for its practice. However, those of skill in the art will, in light of the present disclosure, appreciate that many changes can be made in the specific embodiments that are disclosed and still obtain a like or similar result without departing from the spirit and scope of the invention.

### EXAMPLE I

### Preparation of Promethazine Enantiomers

Although various methods may be used to prepare promethazine enantiomers, the present example particularly describes methods determined to function well in the resolution and preparation of promethazine enantiomers.

In the promethazine base conversion, 100 ml ether and 25 ml 2 M sodium hydroxide (0.045 mol) was added to promethazine hydrochloride (12.5 g, 0.039 mol; Sigma (lot #128H1474). The resulting suspension was shaken and the ether layer was collected. The aqueous layer was extracted twice with ether. The combined ether layers were dried over magnesium sulfate. Rotary evaporation gave 10 g (0.035 mol) promethazine. Yield 90% (step 1).

To prepare promethazine-D-tartrate, promethazine (10 g, 0.035 mol) dissolved in 80 ml acetone was heated in a 60° C. bath while dibenzoyl-D-tartaric acid (12.789 g, 0.036 mol) was added. The resulting clear yellow solution was left at ambient temperature for 3 days (step 2). A heavy precipitate formed, which was filtered off and recrystalized from ethanol four times to give 4.0 g promethazine dibenzoyl-D-tartrate white crystals (step 3).

Promethazine-D-tartrate was converted to promethazine by reaction with sodium hydroxide aqueous solution in ether. The ether layer was separated. The aqueous layer was extracted with ether and the combined ether layer was dried over magnesium sulfate. Rotary evaporation gave 1.6 g promethazine (step 4).

(−)-Promethazine hydrochloride was obtained by precipitation of promethazine with 2 M HCl/ether. After vacuum drying 1.34 g off-white powder was obtained (step 5).

To prepare promethazine-L-tartrate, 11.3 g of brownish liquid was obtained from the acetone mother liquor (Step 2) after rotary evaporation. Similar to Step 4, this liquid was converted to promethazine 3.6 g (step 6). To this 3.6 g of promethazine, 36 ml acetone was added, heated in a 60° C. bath and 4.6040 g dibenzoyl-L-tartaric acid was added. The resulting clear solution was left at ambient for 3 days (step 7).

A heavy precipitate formed, which was filtered off and recrystalized (three times from ethanol, once from acetone, and once more from ethanol) to give 1.2 g promethazine dibenzoyl-L-tartrate white crystals (step 7). Similar to Step 4, promethazine-L-tartrate was converted to promethazine (step 9).

(+)-Promethazine hydrochloride was obtained by precipitation of promethazine with 2 M HCl/ether. After vacuum drying, 0.48 g of off-white powder was obtained (purity 99.87% by HPLC) (step 10).

Repeating Steps 1-5 with 5.7703 g promethazine gave approximately 0.95 g (−)-promethazine hydrochloride as an off-white powder (purity 99.82% by HPLC). X-ray of the promethazine racemate and enantiomers shows that the pure enantiomers are different crystal forms than the racemate. Optical rotation was measured at 27° C. in water.

### EXAMPLE II

### Preparation of Additional Phenothiazine Enantiomers

The present example describes methods determined to function well in the resolution and preparation of additional promethazine enantiomers.

Trimeprazine (TPZ), obtained from Sigma as the racemate, was resolved by preparative column chromatography using CHIRALCEL® OJ-H® preparative column, eluting with 99.9% methanol/0.1% diethylamine at room temperature.

Ethopropazine (EPZ), obtained from Sigma as the racemate ethopropazine hydrochloride, was resolved using the procedures in Example I.

In other studies, racemic ethopropazine hydrochloride salt was mixed with methylene chloride and 2 M sodium hydroxide. The resulting suspension was agitated and the organic layer collected. After drying, the solvent was removed by rotary evaporation to give racemic ethopropazine base (4.0 g, 0.013 mol) that reacted with dibenzoyl-D-tartaric acid (4.4 g, 0.012 mol) in acetone with agitation. A white precipitate was collected after a few hours. After two recrystallization steps from absolute ethanol, a 99+% crystal was obtained, which was converted to ethopropazine hydrochloride salt, Yield: 20%. From the mother liquor, another diastereomeric salt was obtained as white precipitate, which was also recrystalized twice from absolute ethanol before converting to hydrochloride salt. Yield: 20%.

Chiral HPLC chromatograms were obtained from the recrystalized salts. One of the recrystalized salts was determined by HNMR to be the (−)-enantiomer of ethopropazine HCl. The other recrystalized salt was determined by HNMR to be the (+)-enantiomer of ethopropazine HCl.

### EXAMPLE III

### Inhibition of Histamine Activity by Promethazine Enantiomers

This example demonstrates that the major anti-histamine activity associated with promethazine resides in the (+) enantiomer.

H₁ receptor antagonist activity can be measured by a reduced production of IL-6 in controlled studies, for example, as described by Delneste et al., (1994), specifically incorporated herein by reference. In the present studies, HUVEC cells were plated and grown to confluence in 6-well plates. At confluence, the cells were treated with either histamine (₁₀⁻⁴ M); promethazine racemate (10⁻⁵ M) and histamine (10⁻⁴ M); promethazine (+) enantiomer (10⁻⁵ M) and histamine (10⁻⁴ M); promethazine (−) enantiomer (10⁻⁵ M) and Histamine (10⁻⁴ M) or left untreated (U/T) for 5 hours. The total RNA was isolated using Tri-reagent and subjected to reverse transcription polymerase chain reaction (RT-PCR™) analysis of IL-6 production using semi-quantitative analysis against HPRT expression (control gene).

IL-6 was produced by the HUVEC cells endogenously. Histamine alone stimulated an approximately 50% increase in IL-6 mRNA production. Promethazine racemate inhibited histamine stimulation of IL-6 production by 50% of that of cells stimulated only with histamine (i.e., approximately equal to the untreated cells). The (−) enantiomer also produced an approximate 50% reduction in IL-6 production, i.e., essentially the same as that observed for the cells treated with the promethazine racemate. The (+) enantiomer, on the other hand, reduced IL-6 production to 90% of the histamine stimulated cells. These data therefore demonstrate that the major antihistamine activity associated with the promethazine moiety resides in the (+) enantiomer.

### EXAMPLE IV

### Inhibition of Histamine Activity by Additional Phenothiazine Enantiomers

The present example concerns the anti-histamine activity associated with additional phenothiazine enantiomers. In particular, the example demonstrates that the major anti histamine activity associated with ethopropazine resides in the (−) enantiomer.

The IL-6 production assay using HUVEC cells was performed as described in Example III, using the ethopropazine racemate, (+) enantiomer and (−) enantiomer at 10⁻⁵ M and 10⁻⁶ M. At the concentration of 10⁻⁵ M, the anti-histamine activity of each of the racemate, enantiomer and (−) enantiomer was so effective that resolution of the more active enantiomer could not be determined. However, conducting the assays using ethopropazine concentrations of 10⁻⁴ M allowed identification of the more active enantiomer.

HUVEC cells produced IL-6 endogenously. Histamine alone stimulated an approximately 50% increase in IL-6 MRNA production. Ethopropazine racemate reduced the histamine-stimulated production of IL-6 to approximately one fifth of that observed with histamine stimulation. The (+) enantiomer was less active than the racemate, reducing the histamine-stimulated production of IL-6 to approximately two fifths of that observed with histamine stimulation (i.e., just less than that observed in the untreated cells). The (−) enantiomer, on the other hand, reduced IL-6 production to one tenth of that observed with histamine stimulation. That is, the (−) enantiomer was approximately twice as effective as the racemate and four times as effective as the (+) enantiomer. These data therefore demonstrate that the major antihistamine activity associated with ethopropazine resides in the (−) enantiomer.

The same type of IL-6 production assays were also performed using the trimeprazine racemate, (+) enantiomer and (−) enantiomer at 10⁻⁵ M. At the concentration of 10⁻⁵ M, the anti-histamine activity of each of the racemate, (+) enantiomer and (−) enantiomer was so effective that resolution of the more active enantiomer could not be determined. However, conducting the assays using lower ethopropazine concentrations, such as 10⁻⁶ M or lower, will resolve which is the more active enantiomer, as was the case in the ethopropazine studies.

### EXAMPLE V

### High Efficacy Osteoclast Inhibition by the (+) Enantiomer of Promethazine

The present example describes the finding that the ability of promethazine to inhibit osteoclasts, and thus reduce bone resorption, resides in the (+) enantiomer. The example particularly describes the surprising finding that the (+) enantiomer of promethazine has a threefold higher efficacy for osteoclast inhibition than both the racemate and the (−) enantiomer.

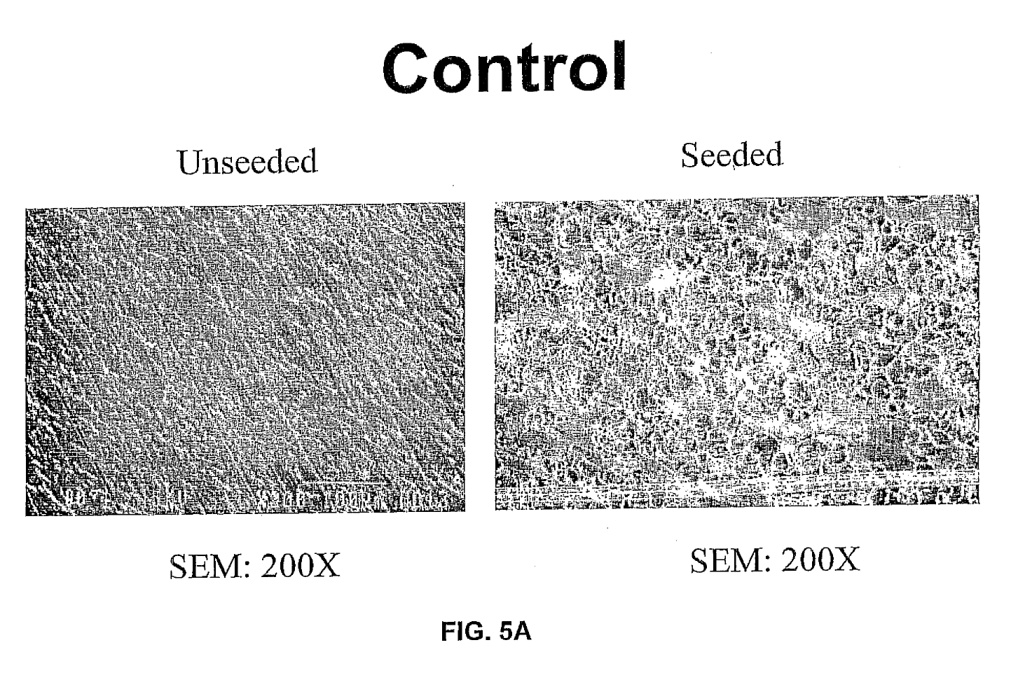

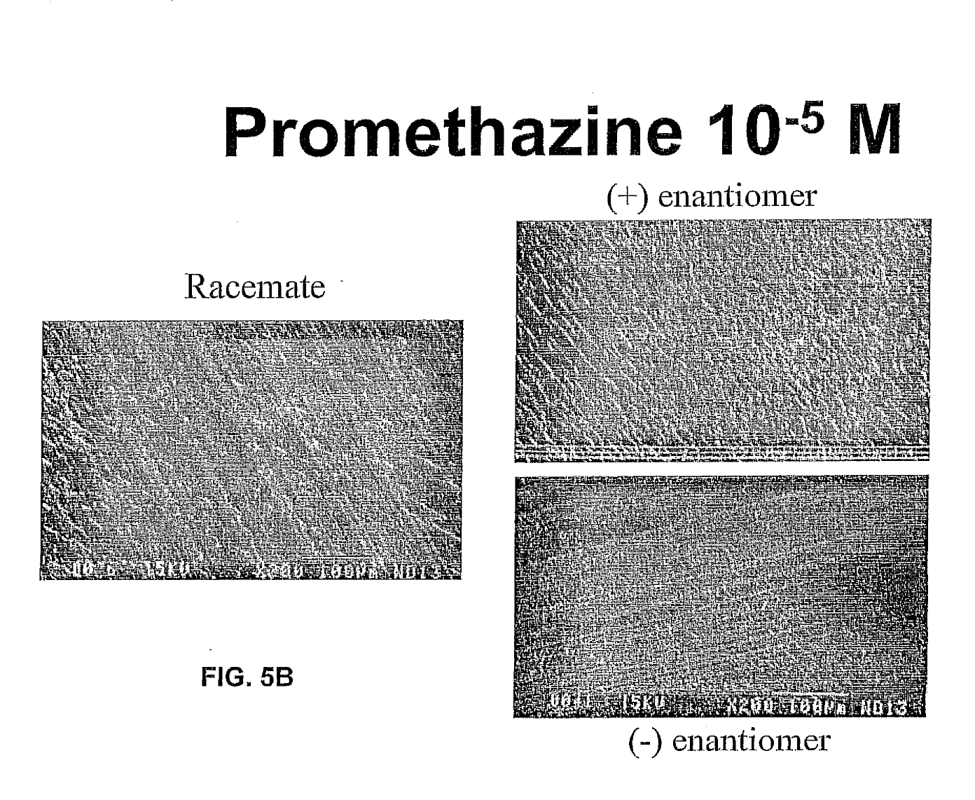

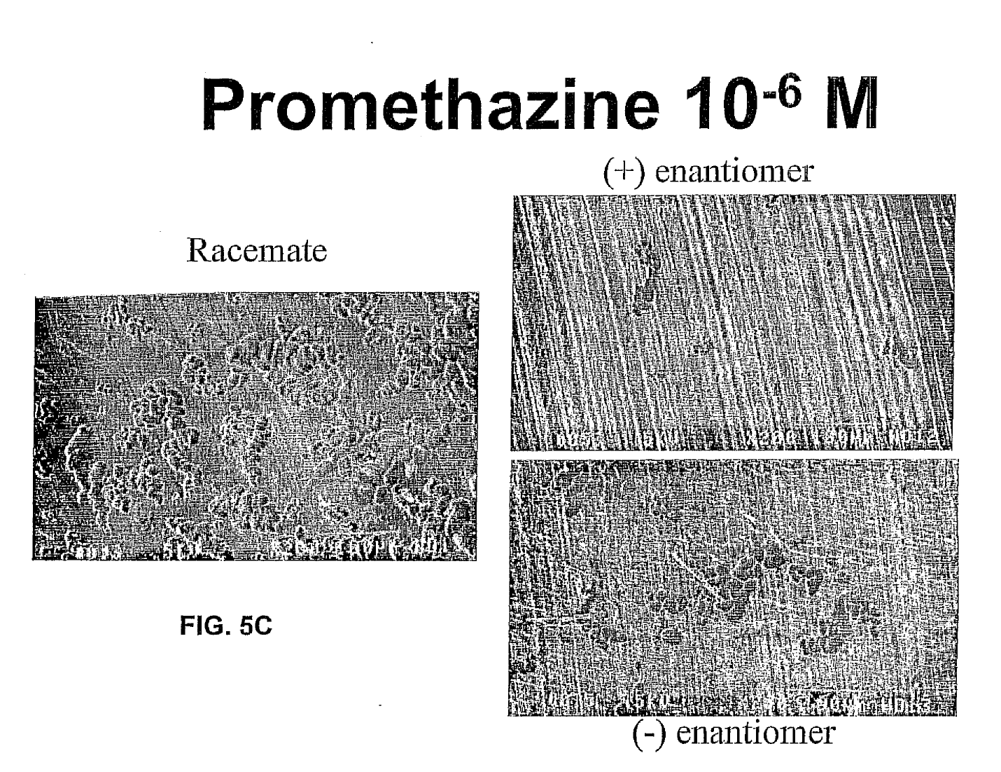

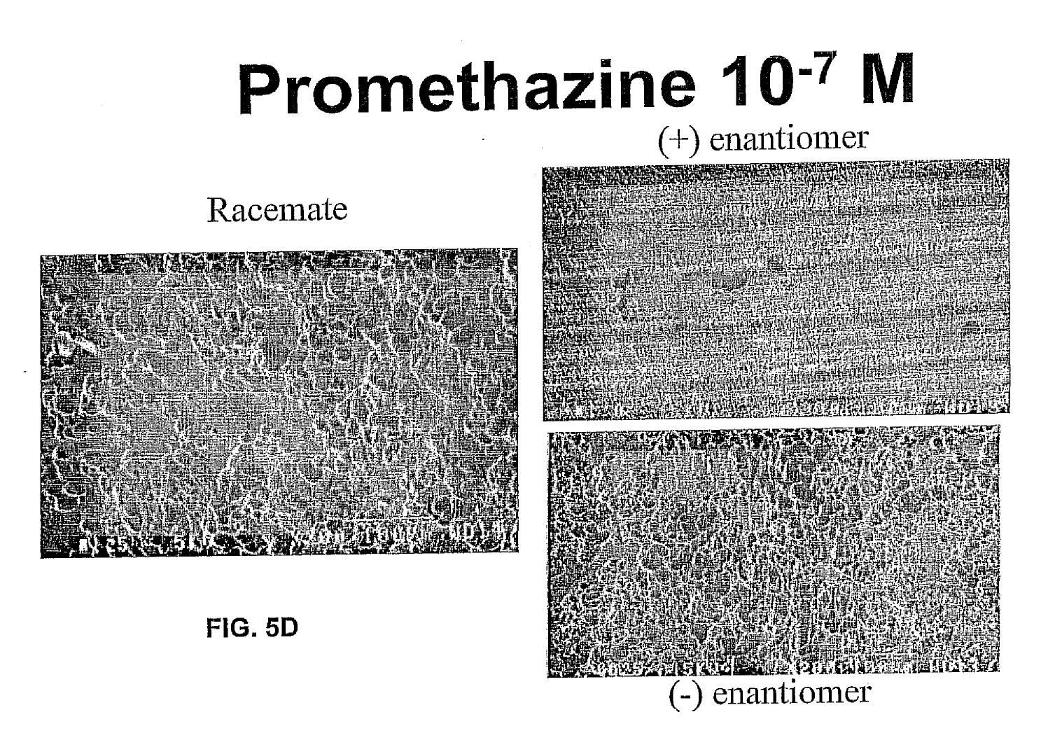

In the osteoclast resorption assay, preosteoblasts are seeded on mammoth tusk slices and incubated in the presence of RANKL and CSF for 14 days with the addition of the respective promethazine enantiomer or racemic mixture at 10⁻⁵ M, 10⁻⁶ M or 10⁻⁷ M (FIG. 5A, FIG. 5B, FIG. 5C and FIG. 5D). During this time, osteoclasts mature and degrade bone matrix. At the end of the incubation period, cells are removed, the specimen examined by SEM and the absorption area/slice is quantitated versus control (untreated) slices.

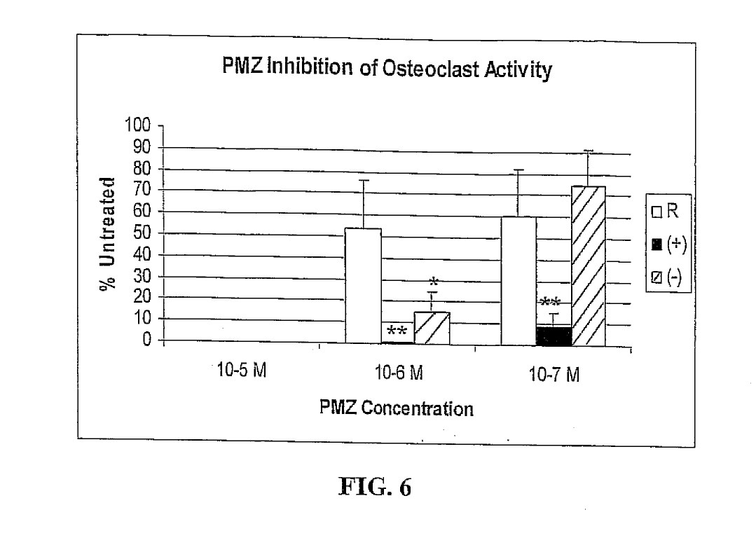

As can be observed visually in FIG. 5A, FIG. 5B, FIG. 5C and FIG. 5D, the (+) enantiomer of promethazine has a higher efficacy for osteoclast inhibition than the racemate and the (−) enantiomer. This data was quantified, wherein the ability of the (+) enantiomer to inhibit osteoclasts was shown to be statistically significant (FIG. 6).

For treatment of osteoporosis, a 90% inhibition of bone resorption is thus achieved with a (+) promethazine enantiomer concentration of 10⁻⁷ M. This is two orders of magnitude lower than the dosage needed for a 90% inhibition using the racemate. Significant inhibition of bone loss in postmenopausal human females was achieved using a 50 mg/day oral dose regimen (Tyan, 1993). Therefore, an exemplary daily dose of the enantiomer is nominally 0.5 mgs/day, even without compensating for the known low bioavailability of an oral route. This dose is well below the dose at which any side effects would occur and thus provides a simple remedy for reducing or eliminating bone loss.

### EXAMPLE VI

### Delivery Devices

This example describes certain resorbable and non-resorbable implantable matrices or transdermal delivery devices that may be used to administer phenothiazine and (+) promethazine enantiomers.

Promethazine inhibits bone loss when administered as the racemate at 50 mg/day (Tyan, 1983). Ethopropazine inhibits the tremors symptomatic of Parkinson's disease at doses as high as 600 mg/day (Brocks, 1999a). At these doses, the side effects include drowsiness, dry mouth, blurred vision and constipation, all likely due to the antihistaminic and/or anticholinergic activity of these compounds. It is also recognized that the phenothiazines are highly protein bound (90-95%), rapidly metabolized to the inactive sulfoxide and other inactive metabolites, and poorly orally bioavailable (Brocks, 1999b).

The use of the more active and single enantiomer of the phenothiazine or promethazine facilitates the use of lower doses to achieve therapeutic efficacy (present Examples, particularly Example V). However, even lower doses can be used to achieve a low and constant drug titer, resulting in significant therapeutic effects with significantly less side effects.

For treatment of osteoporosis, an exemplary daily dose of a (+) promethazine enantiomer is about 0.5 mgs/day by the oral route, which is well below the dose at which any side effects would occur (present Examples, particularly Example V). Release from an implantable controlled release system, as described herein, provides phenothiazine and promethazine enantiomers active at 1/10 the daily dose, based upon the current poor oral bioavailability of the compounds.

In such release systems, non-resorbable or resorbable materials are used to encapsulate the active agent for implantation or for depot use. Examples of non-resorbable materials include polyethylene copolyvinylacetate (EVA) and silicone. Resorbable materials include polylactidecoglycolydes (PGLAs), such as EcoPLA®, polycaproiactones, oxidized regenerated and others. Exemplary non-resorbable polymers are EVA fibers, as in U.S. Pat. No. 4,883,666; U.S. Pat. No. 5,801,835 and U.S. Pat. No. 5,114,719. Fiber implants such as these can be used for multi-month delivery of the optically active phenothiazines and analogues. The compounds can also be delivered in a transdermal patch format, such as the Nycoderm® or Evista® systems.

### EXAMPLE VII

### Synthesis of Additional Phenothiazines

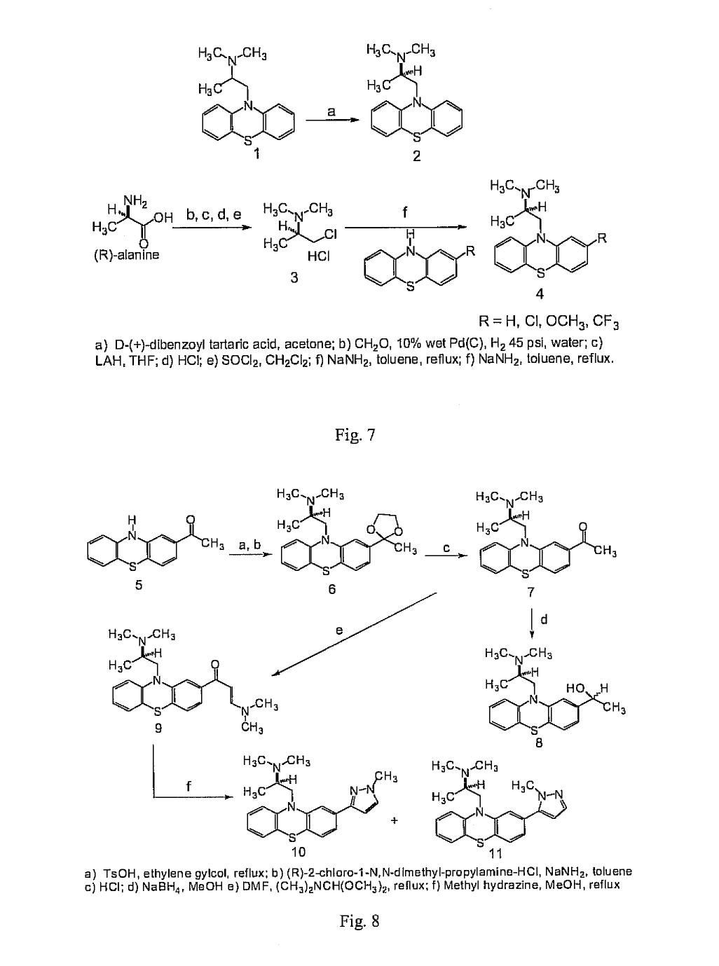

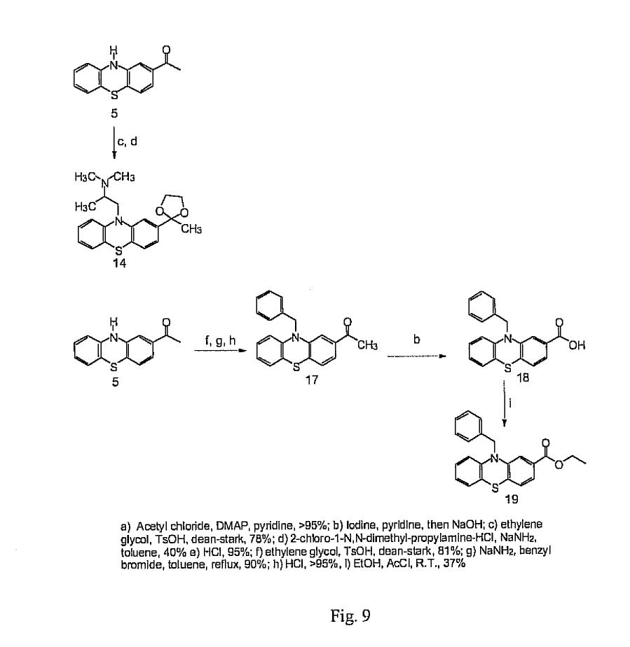

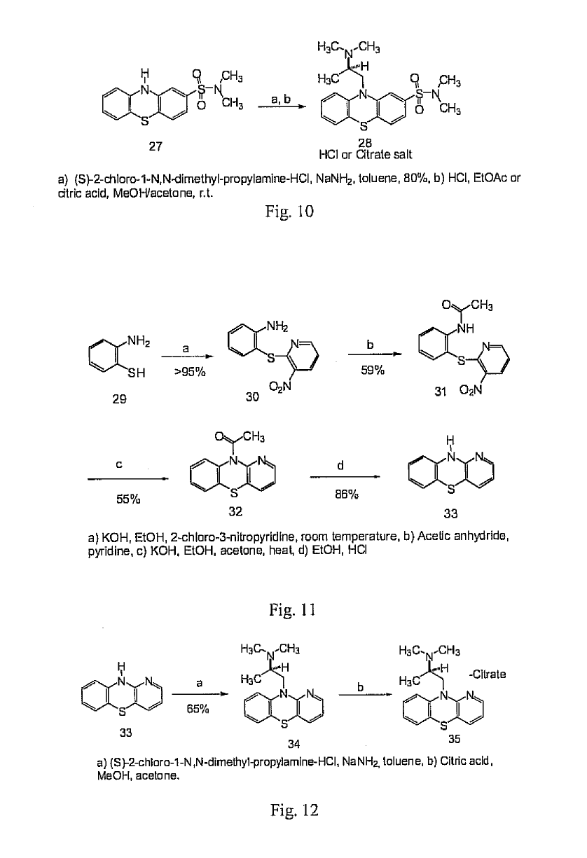

Additional inventive compounds maintaining similar structure to PMZ are designed to possess lower lipophilicity (cLogP or LogD) compared to promethazine as a method of decreasing CNS penetration. Furthermore, intermediates that are screened for activity, as well as used as precursors for future analogs, were prepared on larger scale to ensure sufficient supplies. The synthesis started as shown in schemes I and II of FIGS. 7 and 8, respectively, targeting analogs possessing various polar and non-polar functional groups at the 2-position of the phenothiazine ring. As shown in scheme I, the standard compound for testing is prepared, (+)-Promethazine 2, which is prepared by a known chiral salt resolution utilizing D-(+)-dibenzoyl tartaric acid (Nillson, 1984). In order to avoid the resolution process for every analog prepared, a synthesis of the optically pure (R)-side chain 3 is developed, which can then be incorporated into the phenothiazine core, thus providing direct synthesis of single enantiomer compounds (Kumada, 1983). This four-step synthesis of 3 included: reductive amination of (R)-alanine with formaldehyde, lithium aluminum hydride reduction of the acid and HCl salt formation, and finally, generation of the chloride with thionyl chloride. The chloride 3 could then be utilized in the alkylation step (sodium amide, toluene) to produce the desired compounds of general structure 4. Inventive compounds with substitution at the 2-position are prepared via these methods, as highlighted below by the —OCH₃, Cl and CF₃ analogs.

However, in the cases where the functional handle at the 2-position could be compromised under the sodium amide alkylation conditions, an alternative synthetic route (scheme II) is employed to avoid possible complications. For example, the methyl ketone at the 2-position of the phenothiazine derivative 5 requires protection prior to the alkylation step. Compound 5 is treated with ethylene glycol and catalytic p-toluenesulfonic acid under Dean-stark conditions to produce the intermediate cyclic ketal, which is then alkylated as before with the chloride 3 (scheme I) to produce the desired ketal 6. Deprotection of 6 with HCl cleanly produces the methyl ketone 7. Compound 7 is reduced with sodium borohydride to produce the secondary alcohol 8. The ketone 7 also functions as a synthetic handle for small heterocyclic moieties as well. Treatment of 7 with dimethylformamide-dimethylacetal in DMF produces the corresponding vinylogous amide 9. Cyclization with methyl hydrazine cleanly produces a mixture of the pyrazole isomer 10 and 11. These isomers are easily separated by silica gel chromatography.

Inventive compounds bearing the acid group (CO₂H), amide group (CONR₂), cyano group (CN) and amino groups (NR2) at the 2-position are also prepared. Some of the chemistry attempted to access these analogs is highlighted in scheme III of FIG. 9.

An inert benzyl-protecting group that is stable and does not contain heteroatoms is used. The process for making this precursor involves protection of the ketone as the ketal, alkylation with benzyl bromide and deprotection of the ketal with HCl produced the N-benzyl substrate 17 in good yield. Ketone 17 is then subjected to the same iodoform conditions as discussed above and a crude material, believed to consist of mainly acid 18, is obtained. The compound 18 is treated with HCl/EtOH to afford the ethyl ester 19. Purification of this esterification reaction produces the desired ethyl ester 19.

To assess the effect of a polar sulfonamide group at the 2-position, the phenothiazine 27 is alkylated under the standard conditions shown in Scheme I of FIG. 7 to afford the corresponding sulfonamide. Salt formation with either HCl or citric acid produced the desired sulfonamide analog 28.

Inventive heteroatom-containing analogs are also synthesized (Saraf, 1987). The introduction of the nitrogen into the phenothiazine ring system lowers the cLogP (increases polarity) of these compounds similar to the sulfonamide and acid analogs above. However, unlike the sulfonamide or acid analogs, these aza-promethazine analogs introduce limited structural change to the molecule itself, which does not impact osteoclast activity. The synthesis strategy to access these analogs is shown below in FIGS. 11 and 12, and follows a known literature procedure (Saggiomo, 1958). As shown in FIG. 11, 2-Amino-thiophenol 29 is treated with ethanolic KOH, followed by 2-chloro-3-nitropyridine to afford the desired thio-ether 30 as a brick-red solid. Acetylation with acetic anhydride in pyridine produced the amide derivative 31 in 59% isolated yield. Treatment of the amide 31 with KOH and EtOH produces the desired product 32 via a Smiles-rearrangement process. Deprotection of the acetamide with hot HCl and EtOH produced the desired aza-phenothiazine core 33.

With compound 33 in hand, alkylation reactions are carried out to prepare the desired analogs for screening. As shown below in scheme VII, amine 33 is treated with (S)-2-chloro-N,N-dimethylpropylamine HCl (preparation shown in scheme I) and sodium amide to produce the desired amine 34.

### EXAMPLE VIII

### Osteoclast Screening of Inventive Compounds

A CalciFluor assay is used to measure the calcium that is released as a result of osteoclast-mediated resorptive activity. The first run of this assay looked at three new inventive compounds as well as establishing the data on the internal controls, (+/−)-promethazine and Alendronate (bis-phosphonate). The preliminary data on standards and inventive analogs is provided in Table 3, some general SAR (structure-activity relationships) trends are apparent.

TABLE 3
Inventive Compound OsteoAssay Data

| | | LogP | | OsteoAssay |
|---|---|---|---|---|
| Name | Structure | (lipophilicity) | MW | IC50 (uM) |
| (R)-N,N-dimethyl-l- (10H-phenothiazin-10- yl)propan-2-amine, [(+)-PMZ] | 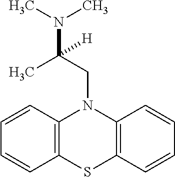 | 3.9 | 284.4 | <1 uM |
| (R)-l-(2-methoxy-10H- phenothiazin-10-yl)- N,N-dimethylpropan-2- amine | 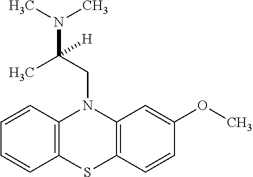 | 3.77 | 314.5 | <1 uM |
| (R)-l-(2-chloro-10H- phenothiazin-10-yl)- N,N-dimethylpropan-2- amine | 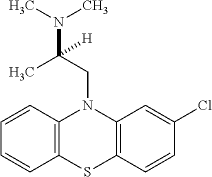 | 4.46 | 318.9 | >10 uM |
| (R)-N,N-dimethyl-1-(2- (trifluoromethyl)-10H- phenothiazin-10- yl)propan-2-amine | 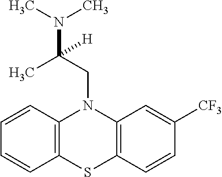 | 4.82 | 352.4 | >10 uM |

The assay is optimized and dose-response data is generated on the Alendronate and Promethazine controls. As shown in FIG. 13, the bis-phosphanate compound Alendronate shows a good dose response with the IC₅₀ in the 10 uM range. This result is very consistent with the observed IC₅₀ of Alendronate in the osteoclast pit assay as well.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps and/or in the sequence of steps of the methods described herein, without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Barros et al., J. Dent. Res., 82(10):791-795, 2003.
Brocks, J. Pharm. Pharmaceut. Sci., (2):39-48, 1999a.
Brocks, Biopharm. Drug. Dispos., 20:159-163, 1999b.
Delneste et al., Clin. Exp. Immunol., 98:344-349, 1994.
Dobigny and Safar, J. Cell. Physiol., 173:10-18, 1997.
Hall and Schaueblin, Calcif Tissue Int., 55:68, 1994.
Jilka et al., J. Clin. Invest., 104:439-446, 1999.
Kumada et al., J. Org. Chem., 48:2195-2202, 1983.
Nilsson et al., Accta Pharm. Suec., 21(5), 309-16, 1984.
Ponder et al., Journal of Chromatography A, 692:173-182, 1995.
Rico et al., Calcif Tissue Int., 65:272-275, 1999.
Saggiomo, A. et al, J. Org. Chem. 23(12), 1906-1909, 1958
Saraf et al., Heterocycles, 26:239-273, 1987.
Schaeublin et al., Gen. Pharmac., 27:845, 1996.
Tani-Ishii et al., J. Periodontol., 74:603-609, 2003.
Tyan, J. Intern. Med, 234:143-149, 1993.
Wang et al., J. Sep. Sci., 24:658-62, 2001.
U.S. Pat. No. 4,256,743

1. A compound having the structure: 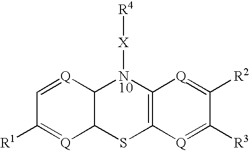
where
R¹, R², and R³ are each independently H, F, Cl, Br, I, C₁-C₆ alkyl, C₁-C₆ fluoro-alkyl, C₁-C₆ perfluoro-alkyl, (C₀-C₆ linear alkyl or C₀-C₆ branched alkyl)-O—R⁶, (C₀-C₆ linear alkyl or C₀-C₆ branched alkyl)-R⁶—OH, (C₀-C₆ linear alkyl or C₀-C₆ branched alkyl)-C(O)OR⁶, —C(O)—R⁶ alkyl, —(C₀-C₆ linear alkyl or C₀-C₆ branched alkyl)-P(O)—(O—R⁶)2, (C₀-C₆ linear alkyl or C₀-C₆ branched alkyl)-S(O)₂—R⁶, (C₀-C₆ linear alkyl or C₀-C₆ branched alkyl)-N(R⁶)₂, (C₀-C₆ linear alkyl or C₀-C₆ branched alkyl)-N⁺(R⁶)₃, (C₀-C₆ linear alkyl or C₀-C₆ branched alkyl)-P⁺R₄, (C₀-C₆ linear alkyl or C₀-C₆ branched alkyl)-S⁺(R⁶)₂, 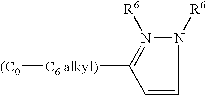
C₆-aryl, a substituted C₆-aryl where the substituent is at least one of hydroxyl radicals, halogens, alkyl radicals having a total of from 1 to 6 carbon atoms, cyclic alkylene groups and heterocyclic alkylene groups having a heterocyclic element of nitrogen and sulfur where C₀ alkyl denotes a nullity;
X is a C₁-C₅ linear or branched alkyl or a C₁-C₅ linear or branched alkenyl with the proviso that at least one chiral carbon atom is present in X;
a purified enantiomer of the at least one chiral carbon atom present in X when R² is other than (C₀-C₆ linear alkyl or C₀-C₆ branched alkyl)-S(O)₂—R⁶;
R⁴ is a tertiary amine or thiol radical structure of N—(R⁵)₂ or S—R⁵ wherein R⁵ in each occurrence is independently hydrogen, C₁-C₄ alkyl radicals and C₁-C₄ alkenyl radical having cyclic alkylene groups, or heterocyclic alkylene groups having a heterocyclic element of nitrogen or sulfur;
R⁶ is independently in each occurrence H, C₁-C₄ alkyl and N—(R⁵)₂; and
Q is independently in each occurrence an sp²-hybridized C—R⁶ or a nitrogen atom.

2. The compound of claim 1 wherein said pharmaceutically acceptable carrier is selected from the group consisting of: an implantable matrix and a transdermal delivery device.

3. The compound of claim 1 wherein said pharmaceutically acceptable carrier is a controlled release oral carrier.

4. The compound of claim 1 wherein R⁵ in every occurrence is the alkyl radicals or alkenyl radical.

5. The compound of claim 1 wherein Xis CH₂—C*H(CH₃)—.

6. The compound of claim 5 wherein R⁴ is N—(R⁴)₂.

7. The compound of claim 5 wherein R¹ and R³ are both H, and Q in each occurrence is the sp²-hybridized C—H.

8. The compound of claim 7 wherein R² is O—R⁶ and R⁶ is C₁-C₄ alkyl.

9. The compound of claim 7 wherein R² is F, Cl, Br, or I.

10. The compound of claim 7 wherein R² is C₁-C₆ perfluoro alkyl.

11. A method of preventing or inhibiting a disease or condition comprising administering to a human patient or animal having or at risk of having a disease or condition associated with bone loss a therapeutically effective amount of a medicament consisting essentially of:
the compound of claim 1;
a pharmaceutically acceptable carrier for said purified enantiomer; and
an optional second anti-osteoclastic or anti-osteoporotic agent with the proviso that said optional second anti-osteoclastic or anti-osteoporotic agent is not the other enantiomer of the compound of claim 1.

12. The method of claim 11 wherein said disease or condition is selected from the group consisting of: periodontitis, osteoporosis and Paget's disease.

13. The method of claim 12 wherein the administration step comprises:
impregnating the compound of claim 1 into an implantable matrix; and
implanting said matrix into said human patient or animal,

14. The method of claim 11 wherein the administration step comprises:
impregnating the compound of claim 1 into a transdermal delivery device; and
placing said transdermal delivery device into contact with said patient or animal.

15. The method of claim 11 wherein said second anti-osteoclastic or anti-osteoporotic agent is a phenothiazine.

16. A process for synthesizing a compound having an enantiomerically pure chiral carbon atom extending from a ring nitrogen atom at position 10 comprising:
reacting a molecule containing the chiral carbon atom and a chloride under alkylation conditions with a phenothiazine having the structure: 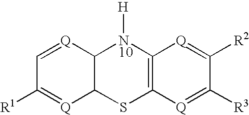
where
R¹, R², and R³ are each independently H, F, Cl, Br, I, C₁-C₆ alkyl, C₁-C₆ fluoro-alkyl, C₁-C₆ perfluoro-alkyl, (C₀-C₆ linear alkyl or C₀-C₆ branched alkyl)-O—R⁶, (C₀-C₆ linear alkyl or C₀-C₆ branched alkyl)-R⁶—OH, (C₀-C₆ linear alkyl or C₀-C₆ branched alkyl)-C(O)OR⁶, —C(O)—R⁶ alkyl, —(C₀-C₆ linear alkyl or C₀-C₆ branched alkyl)-P(O)—(O—R⁶)₂, (C₀-C₆ linear alkyl or C₀-C₆ branched alkyl)-S(O)₂-R⁶, (C₀-C₆ alkyl)-N(R⁶)₂(C₀-C₆ linear alkyl or C₀-C₆ branched alkyl)-N(R⁶)₃, (C₀-C₆ linear alkyl or C₀-C₆ branched alkyl)-P⁺R₄, (C₀-C₆ linear alkyl or C₀-C₆ branched alkyl)-S⁺(R⁶)₂, 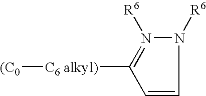
C₆-aryl, a substituted C₆-aryl where the substituent is at least one of hydroxyl radicals, halogens, alkyl radicals having a total of from 1 to 6 carbon atoms, cyclic alkylene groups and heterocyclic alkylene groups having a heterocyclic element of nitrogen and sulfur where C₀ alkyl denotes a nullity;
R⁴ is a tertiary amine or thiol radical structure of N—(R⁵)₂ or S—R⁵ wherein R⁵ in each occurrence is independently hydrogen, C₁-C₄ alkyl radicals and C₁-C₄ alkenyl radical having cyclic alkylene groups, or heterocyclic alkylene groups having a heterocyclic element of nitrogen or sulfur;
R⁶ is independently in each occurrence H, C₁-C₄ alkyl and N—(R⁵)₂; and
Q is independently in each occurrence an sp²-hybridized C—R⁶ or a nitrogen atom;
to produce the compound having the enantiomerically pure chiral carbon atom extending from the ring nitrogen atom at position 10.

17. The process of claim 16 wherein the alkylation conditions are exposure to sodium amide in a solvent.

18. The process of claim 16 wherein the molecule containing the chiral carbon atom is the chloride analog of an amino acid.

19. The process of claim 18 wherein Q is sp²-hybridized C—R⁶ in every occurrence.
